(19) United States
(12) Patent Application Publication
(10) Pub. No.: US 2007/0162983 A1
(43) Pub. Date: Jul. 12, 2007

(54) Diagnostic and therapeutic use of the human sgpl1 gene and protein for neurodegenerative diseases

(75) Inventors: Thomas Hesterkamp, Hamburg (DE); Heinz Von Der Kammer, Hamburg (DE); Johannes Pohlner, Hamburg (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Evotec NeuroSciences GmbH, Hamburg (DE)

(21) Appl. No.: 10/595,930

(22) PCT Filed: Nov. 18, 2004
(86) PCT No.: PCT/EP04/13111

§ 371 Date: May 19, 2006

Related U.S. Application Data
(60) Provisional application No. 60/523,097, filed on Nov. 19, 2003.

(51) Int. Cl. A01K 67/027; C12Q 1/68; G01N 33/53

(52) U.S. Cl. 800/11; 800/14; 435/6; 435/7.1

(57) ABSTRACT

The present invention discloses the differential expression of a gene coding for SGPL1 in specific brain regions of Alzheimer's disease patients. Based on this finding, the invention provides a method for diagnosing or prognosticating a neurodegenerative disease, in particular Alzheimer's disease, in a subject, or for determining whether a subject is at increased risk of developing such a disease. Furthermore, this invention provides therapeutic and prophylactic methods for treating or preventing Alzheimer's disease and related neurodegenerative disorders using the SGPL1 gene and its corresponding gene products. A method of screening for modulating agents of neurodegenerative diseases is also disclosed.

The present invention relates to methods of diagnosing, prognosticating, and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β (Aβ) protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81: 741-66; Greenfield et al., Frontiers Bioscience 2000, 5: D72-83). They are primarily found in the cerebral cortex and hippocampus. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92). The age of onset of AD may vary within a range of 50 years, with early-onset AD occurring in people younger than 65 years of age, and late-onset of AD occurring in those older than 65 years.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). Efforts to detect further susceptibility genes and disease-linked polymorphisms lead to the assumption that specific regions and genes on human chromosomes 10 and 12 may be associated with late-onset AD (Myers et al., Science 2000, 290: 2304-5; Bertram et al., Science 2000, 290: 2303; Scott et al., Am J Hum Genet 2000, 66: 922-32). Although there are rare examples of early-onset AD which have been attributed to genetic defects in the genes for amyloid precursor protein (APP), presenilin-1 and presenilin-2, the prevalent form of late-onset sporadic AD is of hitherto unknown etiologic origin.

The late onset and complex pathogenesis of neurodegenerative disorders pose a formidable challenge to the development of therapeutic and diagnostic agents. It is pivotal to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

The present invention is based on the dysregulation, the differential expression of a gene coding for the sphingosine-1-phosphate lyase 1 (SGPL1), alias sphingosine-1-phosphate lyase or sphinganine-1-phosphate aldolase, and of the protein products in human Alzheimer's disease brain samples. The human SGPL1 cDNA, as referred to in the present invention, was cloned and the corresponding SGPL1 gene was mapped within the AD hot spot region of chromosome 10q (10q22) (Van Veldhoven et al., Biochimica et Biophysica Acta 2000, 1487:128-134; Genbank accession numbers AJ011304, AB033078, AF144638). The cloning of human SGPL1 was based on a search for expressed sequence tags (ESTs) corresponding to the amino acid sequence of the orthologous yeast sphingosine-1-phosphate lyase, encoded by the Saccharomyces cerevisiae BST1/DPL1 gene (Saba et al.; Journal of Biological Chemistry 1997, 272:26087-26090). The SGPL1 gene comprises 15 exons. SGPL1 is ubiquitously expressed in mammalian tissues and cells, except platelets. In humans, mice and rats, SGPL1 expression is highest in the liver, followed by kidney, heart and brain (Van Veldhoven et al., Biochimica et Biophysica Acta 2000, 1487:128-134; Yatomi et al., Journal of Biological Chemistry 1997, 272:5291-5297; reviewed by Pyne, Subcellular Biochemistry 2002, 36:245-268). The human SGPL1 polypeptide (SwissProt accession number 095470) comprises 568 amino acids and is 84% identical to its murine orthologue, which has been proven to restore sphingosine-1-phosphate lyase activity in a BST1/DPL1 gene-deficient yeast strain (Zhou and Saba, Biochemical and Biophysical Research Communications 1998, 242:502-507). Already earlier, the polynucleotide and polypeptide sequences of human SGPL1 and its orthologues in mouse, C. elegans and yeast were described by Saba and Zhou (WO99/16888) and by Duckworth et al. (WO99/38983; U.S. Pat. No. 6,187,562). Human SGPL1 shares 49%, 43%, 42% and 40% amino acid sequence identity with its orthologues in Drosophila melanogaster, Caenorhabditis elegans, Dictyostelium discoideum and Saccharomyces cerevisia. The cysteine residues at positions 218 and 317, the latter being highly conserved, are crucial to human SGPL1 activity. SGPL1 is predicted to be a type-1 transmembrane protein containing one membrane span located near its N-terminus (amino acid positions 39 to 59), which is not required for lyase activity (Van Veldhoven et al., Biochimica et Biophysica Acta 2000, 1487:128-134). SGPL1 is firmly associated with endoplasmic reticulum membranes, while its catalytic site faces the cytoplasm (Van Veldhoven and Mannaerts, Journal of Biological Chemistry 1991, 266:12502-12507). SGPL1, alias sphinganine-1-phosphate aldolase, is a member of the carbon-carbon lyase subclass of aldehyde lyases (EC 4.1.2.27). It requires pyridoxal-5′-phosphate (a vitamin B₆ species) as a co-enzyme. Some specific inhibitors of SGPL1 activity are known, e.g. a 2-vinyl analog of sphinganine phosphate (IC₅₀=2.4 μM), sphinganine-1-phosphonate (Kᵢ=5 μM), the 2D,3L-isomer of sphinganine phosphate (Kᵢ=9.7 μM), and the aminopentol derived from fumonisin B1 by alkaline hydrolysis (IC₅₀=20 μM). Metal ions such as Ca²⁺ and Zn²⁺ inhibit SGPL1 in an unspecific manner. SGPL1 selectively cleaves the C₂-C₃ bond of 1-phosphorylated D-erythro (2D,3D)-isomers of sphingoid bases, most importantly sphingosine-1-phosphate, hereinafter abbreviated S1P (Van Veldhoven, Methods in Enzymology 2000, 311:244-254). SGPL1 is the key enzyme of S1P catabolism. However, putative mechanisms that could regulate SGPL1 expression and/or activity are still unknown. Cellular S1P levels appear to be controlled by its synthesis through sphingosine kinase (S1P) rather than by degradation through SGPL1, S1P phosphatase or LPP1 (Maceyka et al., Biochimica et Biophysica Acta 2002, 1585:193-201; Pyne, Subcellular Biochemistry 2002, 36:245-268). Intracellular S1P concentrations are generally low, except for platelets, which are rich in S1P since they lack SGPL1 activity (Maceyka et al., Biochimica et Biophysica Acta 2002, 1585:193-201; Yatomi et al., Journal of Biological Chemistry 1997, 272:5291-5297). The half-life of S1P in cerebrospinal fluid (CSF) was estimated to be approximately 10 minutes in dogs, and it was suggested that the rapid clearance of S1P from the CSF might reflect rapid degradation and/or uptake into the surrounding tissues and cells due to its amphipathic nature (Tosaka et al., Stroke 2001, 32:2913-2919).

The instant invention discloses that SGPL1 gene expression is dysregulated in AD-affected brains, in that SGPL1 mRNA levels are higher in the temporal cortex and in the hippocampus as compared to the frontal cortex, and higher in the hippocampus as compared to the frontal cortex of AD patients, whereas SGPL1 expression does not differ between the temporal and frontal cortex and between the hippocampus and frontal cortex of healthy age-matched control subjects. SGPL1 is elevated in the temporal cortex but not frontal cortex of AD-patients compared to controls. This dysregulation presumably relates to a pathologic alteration of S1P signaling and homeostasis in AD-affected brains. For instance, it causes increased degradation of S1P in the temporal cortex, resulting in decreased cellular and/or tissue S1P concentrations. This would displace the so called “ceramide/sphingosine-versus-S1P rheostat”, thereby producing a pro-apoptotic imbalance favoring cell death in the affected brain regions which leads to irreversible neuronal damage. In addition, such decreased S1P levels would be insufficient for the maintenance of axonal integrity and/or for the repair of axonal damage resulting, for instance, from oxidative, metabolic, inflammatory and/or other types of stress associated with neurodegenerative diseases, particularly AD. To date, no experiments have been described that demonstrate a relationship between the dysregulation of SGPL1 gene expression and the pathology of neurodegenerative diseases, in particular AD. Likewise, no mutations in the SGPL1 gene have been described to be associated with said diseases. Linking the SGPL1 gene to such diseases offers new ways, inter alia, for the diagnosis and treatment of said diseases.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity or to biological activity and/or pharmacological activity which refers to binding, antagonization, repression, blocking or neutralization. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For the purpose of clarity, a derivative transcript, for instance, refers to a transcript having alterations in the nucleic acid sequence such as single or multiple nucleotide deletions, insertions, or exchanges. A “derivative” may be generated by processes such as altered phosphorylation, or glycosylation, or acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in the biological activity and/or pharmacological activity, in enzyme activity, a change in binding characteristics, or any other change or alteration in the biological, functional, or immunological properties of said translation product of a gene. A “modulator” refers to a molecule which has the capacity to either enhance or inhibit, thus to “modulate” a functional property of an ion channel subunit or an ion channel, to “modulate” binding, antagonization, repression, blocking, neutralization or sequestration of an ion channel or ion channel subunit and to “modulate” activation, agonization and upregulation. “Modulation” will be also used to refer to the capacity to affect the biological activity of a cell. The terms “modulator”, “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. They may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. Such modulators, agents, reagents or compounds can be factors present in cell culture media, or sera used for cell culturing, factors such as trophic factors. “Trophic factors” as used in the present invention include but are not limited to neurotrophic factors, to neuregulins, to cytokines, to neurokines, to neuroimmune factors, to factors derived from the brain (BDNF) and to factors of the TGF beta family. Examples of such trophic factors are neurotrophin 3 (NT-3), neurotrophin 4/5 (NT-4/5), nerve growth factor (NGF), fibroblast growth factor (FGF), epidermal growth factor (EGF), interleukin-beta, glial cell-derived neurotrophic factors (GDNF), ciliary neurotrophic factor (CNTF), insulin-like growth factor (IGF), transforming growth factor (TGF) and platelet-derived growth factor (PDGF). The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. In the art, the terms “identity” and “similarity” mean the degree of polypeptide or polynucleotide sequence relatedness which are determined by matching a query sequence and other sequences of preferably the same type (nucleic acid or protein sequence) with each other. Preferred computer program methods to calculate and determine “identity” and “similarity” include, but are not limited to GCG BLAST (Basic Local Alignment Search Tool) (Altschul et al., J. Mol. Biol. 1990, 215: 403-410; Altschul et al., Nucleic Acids Res. 1997, 25: 3389-3402; Devereux et al., Nucleic Acids Res. 1984, 12: 387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; Needleman and Wunsch, J. Mol. Biol. 1970, 48: 443-453). The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of SGPL1, of SEQ ID NO. 1. “Variants” of a protein molecule include, for example, proteins with conservative amino acid substitutions in highly conservative regions. “Proteins and polypeptides” of the present invention include variants, fragments and chemical derivatives of the protein comprising the amino acid sequences of SGPL1, of SEQ ID NO. 1. Sequence variations shall be included wherein a codon are replaced with another codon due to alternative base sequences, but the amino acid sequence translated by the DNA sequence remains unchanged. This known in the art phenomenon is called redundancy of the set of codons which translate specific amino acids. Included shall be such exchange of amino acids which would have no effect on functionality, such as arginine for lysine, valine for leucine, asparagine for glutamine. Proteins and polypeptides can be included which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules or substances which have been changed and/or that are removed from their natural environment, i.e. isolated from a cell or from a living organism in which they normally occur, and that are separated or essentially purified from the coexisting components with which they are found to be associated in nature, it is also said that they are “non-native”. This notion further means that the sequences encoding such molecules can be linked by the hand of man to polynucleotides to which they are not linked in their natural state and such molecules can be produced by recombinant and/or synthetic means (non-native). Even if for said purposes those sequences may be introduced into living or non-living organisms by methods known to those skilled in the art, and even if those sequences are still present in said organisms, they are still considered to be isolated, to be non-native. In the present invention, the terms “risk”, “susceptibility”, and “predisposition” are tantamount and are used with respect to the probability of developing a neurodegenerative disease, preferably Alzheimer's disease.

The term “AD” shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, New York, 1999; Younkin, Tanzi and Christen, Presenilins and Alzheimer's Disease, Springer Press, Berlin, Heidelberg, New York, 1998). The term “Braak stage” or “Braak staging” refers to the classification of brains according to the criteria proposed by Braak and Braak (Braak and Braak, Acta Neuropathology 1991, 82: 239-259). On the basis of the distribution of neurofibrillary tangles and neuropil threads, the neuropathologic progression of AD is divided into six stages (stage 0 to 6). In the instant invention Braak stages 0 to 2 represent healthy control persons (“controls”), and Braak stages 4 to 6 represent persons suffering from Alzheimer's disease (“AD patients”). The values obtained from said “controls” are the “reference values” representing a “known health status” and the values obtained from said “AD patients” are the “reference values” representing a “known disease status”. Braak stage 3 may represent either a healthy control persons or an AD patient. The higher the Braak stage the more likely is the possibility to display the symptoms of AD. For a neuropathological assessment, i.e. an estimation of the probability that pathological changes of AD are the underlying cause of dementia, a recommendation is given by Braak H. (www.alzforum.org).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Conditions involving neurodegenerative processes are, for instance, age-related macular degeneration, narcolepsy, motor neuron diseases, prion diseases and traumatic nerve injury and repair, and multiple sclerosis.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of a gene coding for SGPL1, and/or of (ii) a translation product of a gene coding for SGPL1, and/or of (iii) a fragment, or derivative, or variant of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease. The wording “in a subject” refers to results of the methods disclosed as far as they relate to a disease afflicting a subject, that is to say, said disease being “in” a subject.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit. Primers for SGPL1 are exemplarily described in Example (iii).

In a further aspect, the invention features a method of monitoring the progression of a neurodegenerative disease in a subject. A level, or an activity, or both said level and said activity, of (i) a transcription product of a gene coding for SGPL1, and/or of (ii) a translation product of a gene coding for SGPL1, and/or of (iii) a fragment, or derivative, or variant of said transcription or translation product in a sample from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of a gene coding for SGPL1, and/or of (ii) a translation product of a gene coding for SGPL1, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said SGPL1 gene, also referred to as sphingosine-1-phosphate lyase 1, or termed sphingosine-1-phosphate lyase or sphinganine-1-phosphate aldolase, or SPL, or EC4.1.2.27, is represented by the sequence of SEQ ID NO.1 (Genbank accession number 095470, which is deduced from the mRNA corresponding to the cDNA sequence of Genbank accession number AB033078), by the sequence of SEQ ID NO.2 and by SEQ ID NO.3 which corresponds to the coding sequence of SGPL1 (SGPL1cds). In the instant invention said sequences are “isolated” as the term is employed herein. Further, in the instant invention, the gene coding for said SGPL1 protein is also generally referred to as the SGPL1 gene, or simply SGPL1, and the protein SGPL1 encoded by the SGPL1 gene is also generally referred to as the SGPL1 protein, or simply SGPL1.

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

The present invention discloses the differential expression, the differential regulation, a dysregulation of a gene coding for SGPL1 in specific samples, in specific brain regions of AD patients and/or in comparison to control persons. Further, the present invention discloses that the gene expression of SGPL1 is varied, is dysregulated in AD-affected brains, in that SGPL1 mRNA levels are up-regulated or elevated in the temporal cortex and/or the hippocampus as compared to the frontal cortex or are down-regulated in the frontal cortex as compared to the temporal cortex and/or the hippocampus. Further, the present invention discloses that the SGPL1 expression differs between the frontal cortex and the temporal cortex and/or the hippocampus of healthy age-matched control subjects compared to the frontal cortex and the temporal cortex and/or the hippocampus of AD patients. No such dysregulation is observed in samples obtained from age-matched, healthy controls. To date, no experiments have been described that demonstrate a relationship between the dysregulation of SGPL1 gene expression and the pathology of neurodegenerative disorders, in particular AD. The link of the SGPL1 gene and the encoded SGPL1 proteins to such diseases, as disclosed in the present invention, offers new ways, inter alia, for the diagnosis and treatment of said disorders, in particular AD.

Neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions and display a selective vulnerability to neuronal loss and degeneration in AD. In contrast, neurons within the frontal cortex, the occipital cortex, and the cerebellum remain largely intact and preserved from neurodegenerative processes. Brain tissues from the frontal cortex (F), the temporal cortex (T), and the hippocampus (H) of AD patients and healthy, age-matched control individuals were used for the herein disclosed examples. Consequently, the SGPL1 gene and its corresponding transcription and/or translation products have a causative role in the regional selective neuronal degeneration typically observed in AD. Alternatively, SGPL1 may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

It is preferred that the sample to be analyzed and determined is selected from the group comprising brain tissue or other tissues, or other body cells. The sample can also comprise cerebrospinal fluid or other body fluids including saliva, urine, serum plasma, blood, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient or healthy control person.

In further preferred embodiments, said reference value is that of a level, or an activity, or both said level and said activity of (i) a transcription product of a gene coding for SGPL1, and/or of (ii) a translation product of a gene coding for SGPL1, and/or of (iii) a fragment, or derivative, or variant of said transcription or translation product in a sample obtained from a subject not suffering from said neurodegenerative disease (healthy control person, control sample, control) or in a sample obtained from a subject suffering from a neurodegenerative disease, in particular Alzheimer's disease (patient sample, patient).

In preferred embodiments, an alteration in the level and/or activity of a transcription product of a gene coding for SGPL1 and/or of a translation product of a gene coding for SGPL1 and/or of a fragment, or derivative, or variant thereof in a sample cell, or tissue, or body fluid from said subject relative to a reference value representing a known health status (control sample) indicates a diagnosis, or prognosis, or increased risk of becoming diseased with a neurodegenerative disease, particularly AD.

In further preferred embodiments, an equal or similar level and/or activity of a transcription product of the gene coding for a SGPL1 protein and/or of a translation product of the gene coding for a SGPL! protein and/or of a fragment, or derivative, or variant thereof in a sample cell, or tissue, or body fluid obtained from a subject relative to a reference value representing a known disease status of a neurodegenerative disease, in particular Alzheimer's disease (AD patient sample), indicates a diagnosis, or prognosis, or increased risk of becoming diseased with said neurodegenerative disease.

In preferred embodiments, measurement of the level of transcription products of an SGPL1 gene is performed in a sample obtained from a subject using a quantitative PCR-analysis with primer combinations to amplify said gene specific sequences from cDNA obtained by reverse transcription of RNA extracted from a sample of a subject. Primer combinations are given in Example (iii) of the instant invention, but also other primers generated from the sequences as disclosed in the instant invention can be used. A Northern blot with probes specific for said gene can also be applied. It might further be preferred to measure transcription products by means of chip-based microarray technologies. These techniques are known to those of ordinary skill in the art (see e.g. Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or activity of a translation product of a gene coding for SGPL1 and/or of a fragment, or derivative, or variant of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Using Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of a gene coding for SGPL1, and/or of (ii) a translation product of a gene coding for SGPL1, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:
(a) at least one reagent which is selected from the group consisting of (i) reagents that selectively detect a transcription product of a gene coding for SGPL1, and (ii) reagents that selectively detect a translation product of a gene coding for SGPL1; and
   (b) an instruction for diagnosing, or prognosticating a neurodegenerative disease, in particular AD, or determining the propensity or predisposition of a subject to develop such a disease by describing the steps of:
   detecting a level, or an activity, or both said level and said activity, of said transcription product and/or said translation product of a gene coding for SGPL1, in a sample obtained from said subject; and
   diagnosing or prognosticating a neurodegenerative disease, in particular AD, or determining the propensity or predisposition of said subject to develop such a disease, wherein a varied or altered level, or activity, or both said level and said activity, of said transcription product and/or said translation product compared to a reference value representing a known health status (control) and/or wherein a level, or activity, or both said level and said activity, of said transcription product and/or said translation product is similar or equal to a reference value representing a known disease status, preferably a disease status of AD, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD.

In a further aspect the invention features the use of a kit in a method of diagnosing or prognosticating a neurodegenerative disease, in particular Alzheimer's disease, in a subject, and in a method of determining the propensity or predisposition of a subject to develop such a disease by the steps of: (i) detecting in a sample obtained from said subject a level, or an activity, or both said level and said activity of a transcription product and/or of a translation product of a gene coding for SGPL1, and (ii) comparing said level or activity, or both said level and said activity of a transcription product and/or of a translation product of a gene coding for SGPL1 to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) a gene coding for SGPL1, and/or (ii) a transcription product of a gene coding for SGPL1, and/or (iii) a translation product of a gene coding for SGPL1, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of a gene coding for SGPL1 protein, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of a gene coding for SGPL1 protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN& P 1992, 5: 389-395; Agrawal and Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against transcripts of a gene coding for SGPL1. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see Mc Celland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mammal, a mouse, a rat, a fish, an insect, or a worm; a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) a gene coding for SGPL1, and/or (ii) a transcription product of a gene coding for SGPL1 and/or (iii) a translation product of a gene coding for SGPL1, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for SGPL1, and/or (ii) a transcription product of a gene coding for SGPL1, and/or (iii) a translation product of a gene coding for SGPL1, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) a gene coding for SGPL1, and/or (ii) a transcription product of a gene coding for SGPL1 and/or (iii) a translation product of a gene coding for SGPL1, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, genetically altered non-human animal comprising a non-native gene sequence coding for SGPL1, or a fragment, or a derivative, or variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular AD. Strategies and techniques for the generation and construction of such an animal are known to those of ordinary skill in the art (see e.g. Capecchi, Science 1989, 244: 1288-1292; Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999). It is preferred to make use of such a recombinant, genetically altered non-human animal, transgenic or knockout animal, as an animal model for investigating neurodegenerative diseases, in particular Alzheimer's disease. Such an animal may be a test animal or an experimental animal useful for screening, testing and validating compounds, agents and modulators in the development of diagnostics and therapeutics to treat neurodegenerative diseases, in particular Alzheimer's disease.

In another aspect, the invention features an assay for screening for a modulator, or an agent, or compound of neurodegenerative diseases, in particular AD, or related diseases and disorders of one or more substances selected from the group consisting of (i) a gene coding for SGPL1, and/or (ii) a transcription product of a gene coding for SGPL1, and/or (iii) a translation product of a gene coding for SGPL1, and/or (iv) a fragment, or derivative, or variant of (i) to (iii). This screening method comprises (a) contacting a cell with a test compound, agent, or modulator and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells, or the contacted cells, indicates that the test compound, or agent, or modulator, is a modulator of said diseases and disorders, wherein said modulator can be the activity, or the level, or both the activity and the level of one or more substances recited in (i) to (iv).

Examples of cells used in said screening assay, such as cells over-expressing the SGPL1 protein, preferably stably over-expressing the SGPL1 protein, as disclosed in the present invention, are given below (Example (v) and FIG. 11). The examples of genetically altered cells as disclosed are illustrative only and not intended to limit the remainder of the disclosure in any way.

In one further aspect, the invention features a screening assay for a modulator of neurodegenerative diseases, in particular AD, or related diseases and disorders of one or more substances selected from the group consisting of (i) a gene coding for SGPL1, and/or (ii) a transcription product of a gene coding for SGPL1, and/or (iii) a translation product of a gene coding for SGPL1, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said symptoms and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal, or experimental animal, or animal model and/or said control animal is a recombinant, genetically altered non-human animal which expresses a gene coding for SGPL1, or a fragment thereof, or a derivative, or a variant thereof, under the control of a transcriptional regulatory element which is not the native SGPL1 gene transcriptional control regulatory element.

In a further aspect, the genetically altered non-human animals according to the present invention provide an in-vivo assay to determine or validate the efficacy of therapies, or modulatory agents, or compounds for the treatment of neurodegenerative diseases, in particular Alzheimer's disease.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and a translation product of a gene coding for SGPL1, or a fragment, or derivative, or variant thereof. Said screening assay comprises the steps of (i) adding a liquid suspension of said SGPL1 translation product, or a fragment, or derivative, or variant thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said SGPL1 translation product, or said fragment, or derivative, or variant thereof, and said compound or plurality of compounds, and said detectable, preferably fluorescently labelled ligand, and (v) measuring the amounts of preferably the fluorescence associated with said SGPL1 translation product, or with said fragment, or derivative, or variant thereof, and (vi) determining the degree of inhibition by one or more of said compounds of binding of said ligand to said SGPL1 translation product, or said fragment, or derivative, or variant thereof. It might be preferred to reconstitute said SGPL1 translation product, or fragment, or derivative, or variant thereof into artificial liposomes to generate the corresponding proteoliposomes to determine the inhibition of binding between a ligand and said SGPL1 translation product. Methods of reconstitution of SGPL1 translation products from detergent into liposomes have been detailed (Schwarz et al., Biochemistry 1999, 38: 9456-9464; Krivosheev and Usanov, Biochemistry-Moscow 1997, 62: 1064-1073). Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of the gene coding for SGPL1, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for the screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a gene product of a gene coding for SGPL1 by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to a translation product of a gene coding for SGPL1, or to a fragment, or derivative, or variant thereof. Said screening assay comprises (i) adding a liquid suspension of said SGPL1 translation product, or a fragment, or derivative, or variant thereof, to a plurality of containers, and (ii) adding a detectable, preferably a fluorescently labelled compound or a plurality of detectable, preferably fluorescently labelled compounds to be screened for said binding to said plurality of containers, and (iii) incubating said SGPL1 translation product, or said fragment, or derivative, or variant thereof, and said detectable, preferably fluorescently labelled compound or detectable, preferably fluorescently labelled compounds, and (iv) measuring the amounts of preferably the fluorescence associated with said SGPL1 translation product, or with said fragment, or derivative, or variant thereof, and (v) determining the degree of binding by one or more of said compounds to said SGPL1 translation product, or said fragment, or derivative, or variant thereof. In this type of assay it might be preferred to use a fluorescent label. However, any other type of detectable label might also be employed. Also in this type of assay it might be preferred to reconstitute a SGPL1 translation product or fragment, or derivative, or variant thereof into artificial liposomes as described in the present invention. Said assay methods may be useful for the identification of novel compounds as well as for evaluating compounds which have been improved or otherwise optimized in their ability to bind to an SGPL1 translation product, or fragment, or derivative, or variant thereof.

In one further embodiment, the present invention provides a method for producing a medicament comprising the steps of (i) identifying a compound as a binder to a gene product of the SGPL1 gene by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule and the use of said protein molecule as shown in SEQ ID NO. 1, said protein molecule being a translation product of the gene coding for SGPL1, or fragments, or derivatives, or variants thereof, as diagnostic target for detecting a neurodegenerative disease, preferably Alzheimer's disease.

The present invention further features a protein molecule and the use of said protein molecule as shown in SEQ ID NO. 1, said protein molecule being a translation product of the gene coding for SGPL1, or fragments, or derivatives, or variants thereof, as screening target for reagents or compounds preventing, or treating, or ameliorating a neurodegenerative disease, preferably Alzheimer's disease.

The present invention features an antibody which is specifically immunoreactive with an immunogen, wherein said immunogen is a translation product of a gene coding for SGPL1, SEQ ID NO. 1, or a fragment, or variant, or derivative thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the SGPL1 gene, or fragments, or derivatives, or variants thereof.

In a preferred embodiment of the present invention, said antibodies can be used for detecting the pathological state of a cell in a sample obtained from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

### FIGURES

FIGS. 1 and 2 illustrate the differential expression of the human SGPL1 gene in AD brain tissues by quantitative RT-PCR analysis. Quantification of RT-PCR products from RNA samples collected from the frontal cortex (F) and the temporal cortex (T) of AD patients (FIG. 1a) and samples from the frontal cortex (F) and the hippocampus (H) of AD patients (FIG. 2a) was performed by the LightCycler rapid thermal cycling technique. Likewise, samples of healthy, age-matched control individuals were compared (FIG. 1b for frontal cortex and temporal cortex, FIG. 2b for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of SGPL1 cDNAs from both, the frontal and temporal cortices of a normal control individual, and from the frontal cortex and hippocampus of a normal control individual, respectively, during the exponential phase of the reaction are juxtaposed (FIGS. 1b and 2b, arrowheads), whereas in Alzheimer's disease (FIGS. 1a and 2a, arrowheads) there is a significant separation of the corresponding curves, indicating a differential expression of the gene coding for SGPL1 in the respective analyzed brain regions, indicating a dysregulation, preferably an upregulation of a transcription product of the human SGPL1 gene, or a fragment, or derivative, or variant thereof, in the temporal cortex relative to the frontal cortex, and in the hippocampus relative to the frontal cortex.

FIG. 3 discloses SEQ ID NO. 1, the full-length amino acid sequence of the human SGPL1 protein, comprising 568 amino acids, as defined by the SwissProt accession number 095470.

FIG. 4 shows SEQ ID NO. 2, the nucleotide sequence of the human SGPL1 cDNA, comprising 5741 nucleotides, as defined by the Genbank accession number AB033078.

FIG. 5 shows the nucleotide sequence of SEQ ID NO. 3, the coding sequence (cds) of the human SGPL1 gene, comprising 1707 nucleotides (nucleotides 201-1907 of SEQ ID NO. 2).

FIG. 6 depicts the sequence alignment of the primers used for SGPL1 transcription level profiling by quantitative RT-PCR with the corresponding clippings of SEQ ID NO. 2.

FIG. 7 shows the analysis of absolute mRNA expression of SGPL1 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of control persons with each other. Said difference reflects an upregulation of SGPL1 in the temporal cortex of AD patients relative to the temporal cortex of control persons which is prominent comparing the Braak stages 0-3 with Braak stages 4-6 with each other. In frontal cortices a comparable upregulation cannot be observed. The differences reflect as well a down-regulation of SGPL1 in the frontal cortex of AD patients compared to the frontal cortex of control group subjects. The Braak stages correlate with the progressive course of AD disease which, as shown in the instant invention, is associated with an increasing difference in the regulation, the level and the activity of SGPL1 as described above.

FIG. 8 lists the SGPL1 gene expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (0.29 to 1.96 fold, values according to the formula described below) and twentyfive healthy, age-matched control individuals, herein identified by internal reference numbers C005, C008, C011, C012, C014, C025, C026, C027, C028, C029, C030, C031, C032, C033, C034, C035, C036, C038, C039, C041, C042, DE02, DE03, DE05, DE07 (0.81 to 19.33 fold, values according to the formula described below). For an -regulation in the temporal cortex, the values shown are calculated according to the formula described herein (see below) and in case of an up-regulation in the frontal cortex the reciprocal values are calculated, respectively. The bar diagram visualizes individual natural logarithmic values of the temporal to frontal cortex, ln(IT/IF), and of the frontal to temporal cortex regulation factors, ln(IF/IT), in different Braak stages (0 to 6).

FIG. 9 lists the gene expression levels in the hippocampus relative to the frontal cortex for the SGPL1 gene in six Alzheimer's disease patients, herein identified by internal reference numbers P010, P011, P012, P014, P016, P019 (1.18 to 1.99 fold) and three healthy, age-matched control individuals, herein identified by internal reference numbers C004, C005, C008 (0.80 to 1.34 fold). The values shown are calculated according to the formula described herein (see below). The scatter diagram visualizes individual logarithmic values of the hippocampus to frontal cortex regulation ratios, log(ratio HC/IF), in control samples (dots) and in AD patient samples (triangles).

FIG. 10 depicts a Western blot image of total human brain extracts labeled with polyclonal anti-myc antibody (MBL, 1.1000).

Lanes A and B: total protein extract of H4APPsw cells stably expressing SGPL1 tagged with a myc-tag (SGPL1-myc). The arrow indicates a major band at about 58 kDa, which corresponds to the predicted molecular weight of the full length SGPL1 protein.

FIG. 11 shows the immunofluorescence analysis of H4APPsw control cells and H4APPsw cells stably over-expressing the myc-tagged SGPL1 protein (H4APPsw-SGPL1cds-myc). The SGPL1-myc protein was detected with rabbit polyclonal anti-myc antibodies (Mobitec) and a Cy3-conjugated anti-rabbit antibody (Amersham) (FIGS. 11A and B). The cellular nucleus was stained with DAPI (FIGS. 11C and D). The overlay analysis indicate that the SGPL1cds-myc protein is localized to the endoplasmatic reticulum and to the membrane (FIG. 11E) and is over-expressed in more than 70% of the H4APPsw-SGPL1cds-myc transduced cells as compared to the H4APPsw control cells (FIG. 11F).

### EXAMPLE I

(i) Brain Tissue Dissection from Patients with AD:

Brain tissues from AD patients and age-matched control subjects were collected on average within 5 hours post-mortem and immediately frozen on dry ice. Sample sections from each tissue were fixed in paraformaldehyde for histopathological confirmation of the diagnosis. Brain areas for differential expression analysis were identified and stored at −80° C. until RNA extractions were performed.

(ii) Isolation of Total mRNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were used to generate a melting curve with the LightCycler technology as described in the manufacturer's protocol (Roche).

(iii) Quantitative RT-PCR Analysis:

The expression levels of the human SGPL1 gene in temporal cortex versus frontal cortex and in the hippocampus versus frontal cortex were analyzed using the LightCycler technology (Roche). This technique features rapid thermal cycling for the polymerase chain reaction as well as real-time measurement of fluorescent signals during amplification and therefore allows for highly accurate quantification of RT-PCR products by using a kinetic, rather than endpoint readout. The ratios of SGPL1 cDNAs from temporal cortices of AD patients and of healthy age-matched control individuals, from the frontal cortices of AD patients and of healthy age-matched control individuals, from the hippocampi of AD patients and of age-matched control individuals, and the ratios of SGPL1 cDNAs from the temporal cortex and frontal cortex of AD patients and of healthy age-matched control individuals, and the ratios of SGPL1 cDNAs from the hippocampus and from frontal cortex of AD patients and of healthy age-matched control individuals, respectively, were determined (relative quantification).

The mRNA expression profiling between frontal cortex tissue (F) and inferior temporal cortex tissue (T) of SGPL1 has been analyzed in four up to nine tissues per Braak stage. Because of the lack of high quality tissues from one donor with Braak 3 pathology, tissues of one additional donor with Braak 2 pathology were included, and because of the lack of high quality tissues from one donor with Braak 6 pathology, tissue samples of one additional donor with Braak 5 pathology were included.

For the analysis of the profiling, two general approaches have been applied. Both comparative profiling studies, frontal cortex against inferior temporal cortex as well as control against AD patients, which contribute to the complex view of the relevance of SGPL1 in AD physiology, are shown in detail below.

1) Relative Comparison of the mRNA Expression Between Frontal Cortex Tissue and Inferior Temporal Cortex Tissue of Controls and of AD Patients.

This approach allowed to verify that SGPL1 is either involved in the protection of the less vulnerable tissue (frontal cortex) against degeneration, or is involved in or enhances the process of degeneration in the more vulnerable tissue (inferior temporal cortex).

First, a standard curve was generated to determine the efficiency of the PCR with specific primers for the gene coding for SGPL1:

5′-TGCCCACTGATACCAAGACCA-3′
(SEQ ID NO. 2, nucleotides 4802-4822)
and
5′-AGTGCCTGGAAATGAGATGGA-3′
(SEQ ID NO. 2, nucleotides 4849-4869).

PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 80.7° C. with no visible primer dimers. Quality and size of the PCR product were determined with the DNA LabChip system (Agilent 2100 Bioanalyzer, Agilent Technologies). A single peak at the expected size of 68 bp for the SGPL1 gene was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTTTGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for SGPL1 and the five reference standard genes. The slopes and the intercepts of the standard curves (i.e. linear regressions) were calculated for all genes. In a second step, cDNAs from frontal cortices of AD patients and of healthy control individuals, from temporal cortices of AD patients and of healthy control individuals, from hippocampi of AD patients and of healthy control individuals, and cDNAs from the frontal cortex and the temporal cortex of AD patients and of control individuals and from the frontal cortex and the hippocampus of AD patients and of control individuals, respectively, were analyzed in parallel and normalized to cyclophilin B. The Cₜ values were measured and converted to ng total brain cDNA using the corresponding standard curves:
10ˆ((Cₜvalue−intercept)/slope)[ng total brain cDNA]

The values for temporal and frontal cortex SGPL1 cDNAs, the values for hippocampus and frontal cortex SGPL1 cDNAs, and the values from the frontal cortex SGPL1 cDNAs of AD patients (P) and control individuals (C), and the values for temporal cortex SGPL1 cDNAs of AD patients (P) and of control individuals (C), were normalized to cyclophilin B, and the ratios were calculated according to formulas:

$$Ratio = \frac{SGPL 1 temporal [ ng ] / cyclophilin B temporal [ ng ]}{SGPL 1 frontal [ ng ] / cyclophilin B frontal [ ng ]}$$

$$Ratio = \frac{\begin{matrix}SGPL 1 hippocampus [ ng ] / \\ cyclophilin B hippocampus [ ng ]\end{matrix}}{SGPL 1 frontal [ ng ] / cyclophilin B frontal [ ng ]}$$

$$Ratio = \frac{\begin{matrix}SGPL 1 ( P ) temporal [ ng ] / \\ cyclophilin B ( P ) temporal [ ng ]\end{matrix}}{\begin{matrix}SGPL 1 ( C ) temporal [ ng ] / \\ cyclophilin B ( C ) temporal [ ng ]\end{matrix}}$$

$$Ratio = \frac{SGPL 1 frontal [ ng ] / cyclophilin B ( P ) frontal [ ng ]}{\begin{matrix}SGPL 1 ( C ) frontal [ ng ] / \\ cyclophilin B ( C ) frontal [ ng ]\end{matrix}}$$

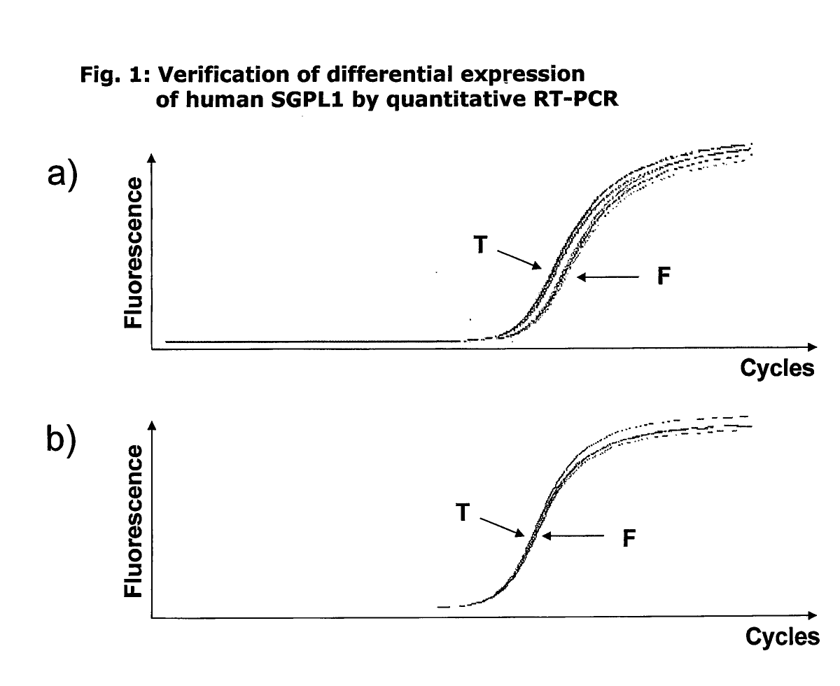

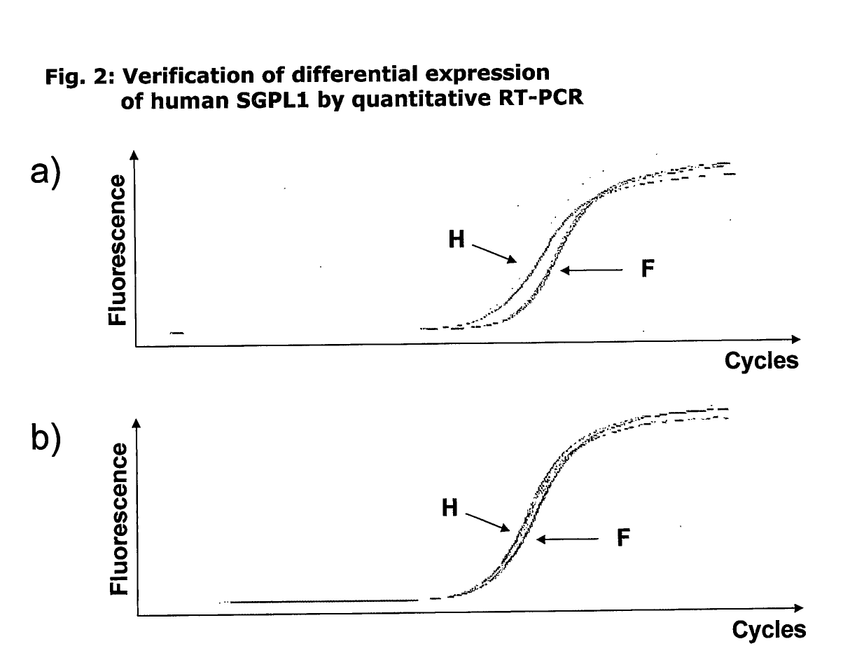

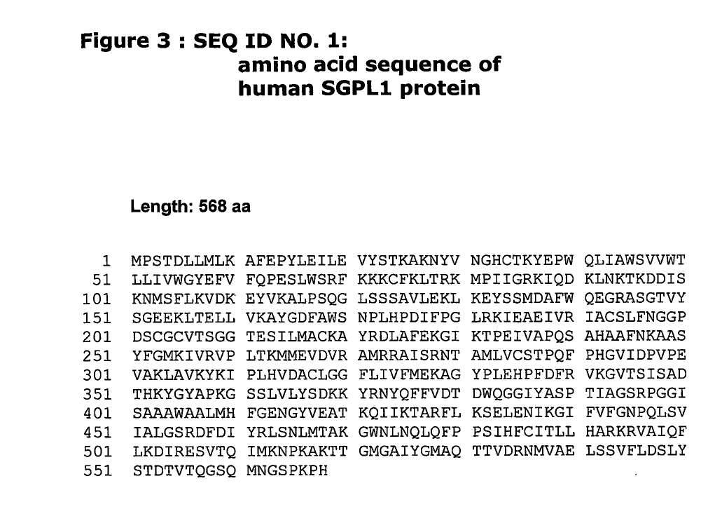

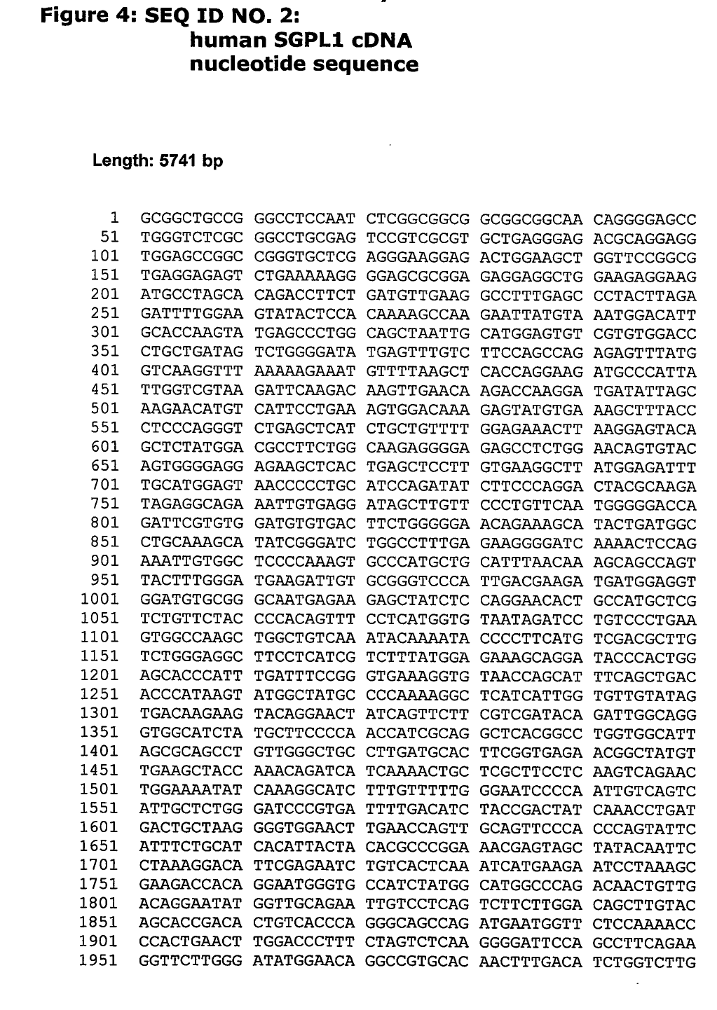

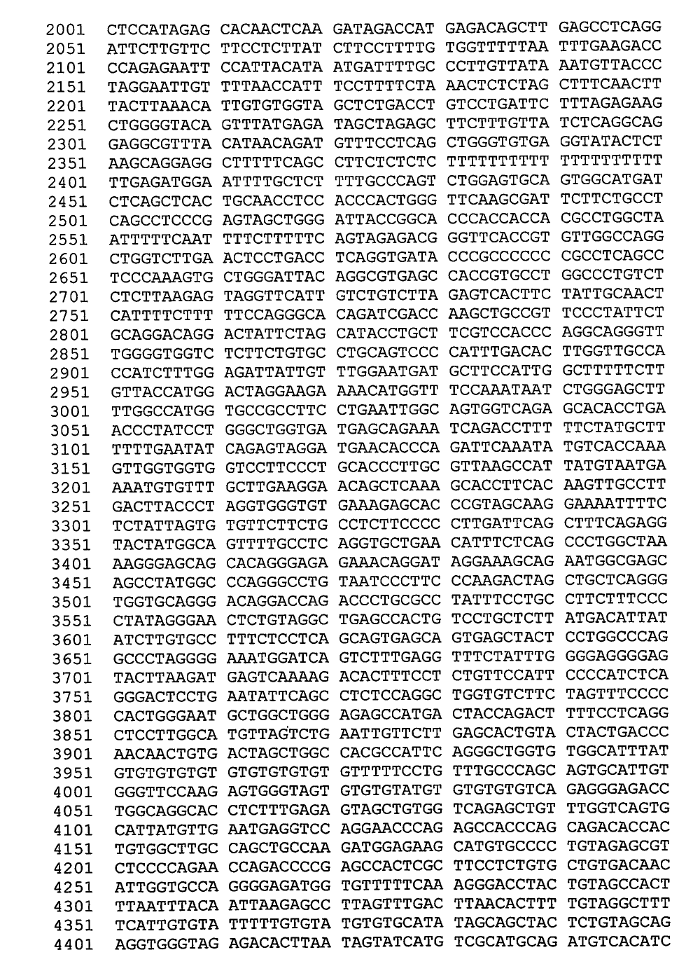

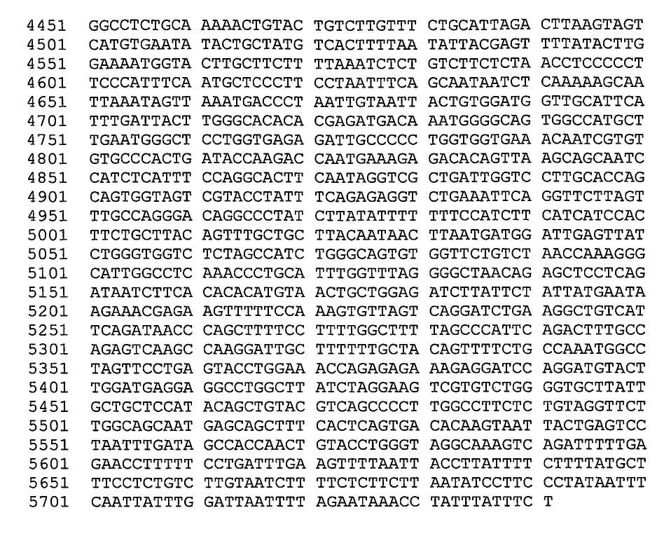

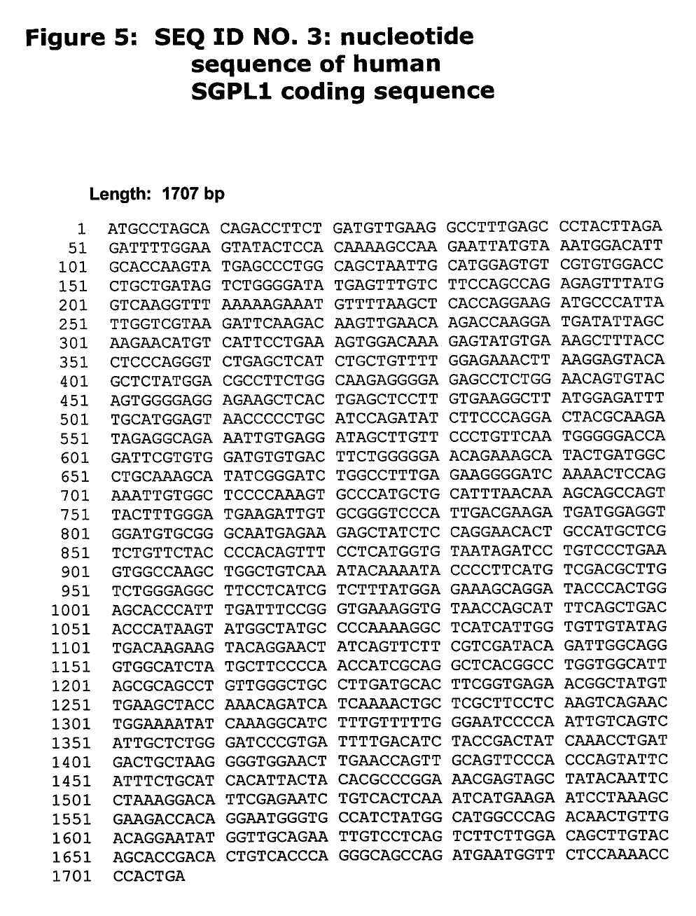

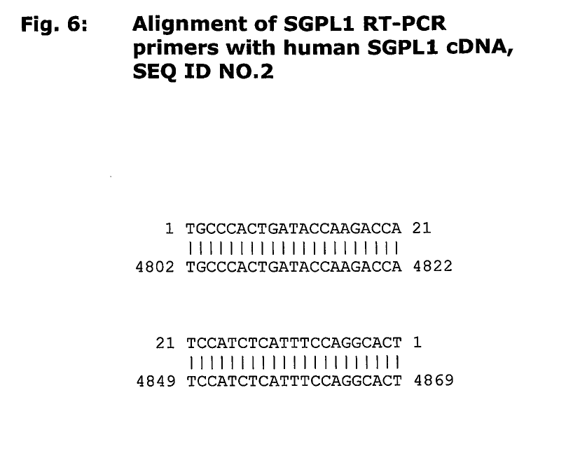

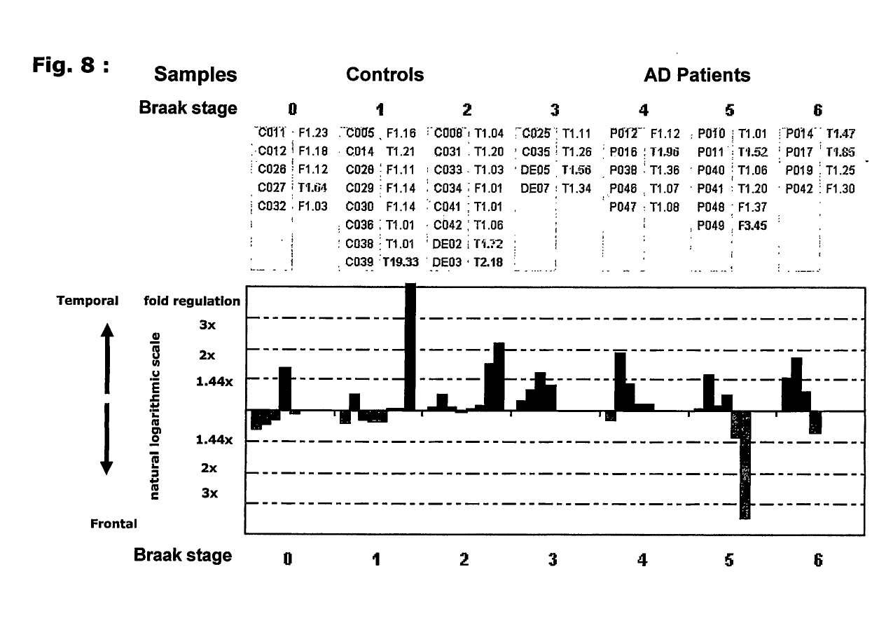

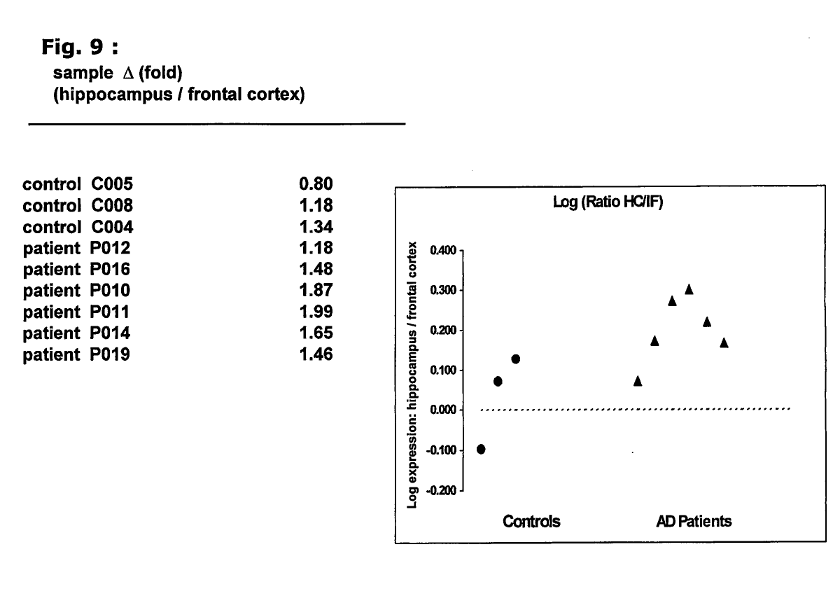

In a third step, the set of reference standard genes was analyzed in parallel to determine the mean average value of the AD patient to control person temporal cortex ratios, of the AD patient to control person frontal cortex ratios, and of the temporal to frontal ratios of AD patients and control persons and the hippocampi to frontal ratios of AD patients and control persons, respectively, of expression levels of the reference standard genes for each individual brain sample. As cyclophilin B was analyzed in step 2 and step 3, and the ratio from one gene to another gene remained constant in different runs, it was possible to normalize the values for SGPL1 to the mean average value of the set of reference standard genes instead of normalizing to one single gene alone. The calculation was performed by dividing the respective ratio shown above by the deviation of cyclophilin B from the mean value of all housekeeping genes. The results of such quantitative RT-PCR analysis for the SGPL1 gene and the respective calculated values for the gene coding for the SGPL1 are shown in FIGS. 1 and 8 and in FIGS. 2 and 9.

2) Comparison of the mRNA Expression Between Controls and AD Patients.

For this analysis it was proven that absolute values of real-time quantitative PCR (Lightcycler method) between different experiments at different time points are consistent enough to be used for quantitative comparisons without usage of calibrators. Cyclophilin was used as a standard for normalization in any of the qPCR experiments for more than 100 tissues. Between others it was found to be the most consistently expressed housekeeping gene in our normalization experiments. Therefore a proof of concept was done by using values that were generated for cyclophilin.

First analysis used cyclophilin values from qPCR experiments of frontal cortex and inferior temporal cortex tissues from three different donors. From each tissue the same cDNA preparation was used in all analyzed experiments. Within this analysis no normal distribution of values was achieved due to small number of data. Therefore the method of median and its 98%-confidence level was applied. This analysis revealed a middle deviation of 8.7% from the median for comparison of is absolute values and a middle deviation of 6.6% from the median for relative comparison.

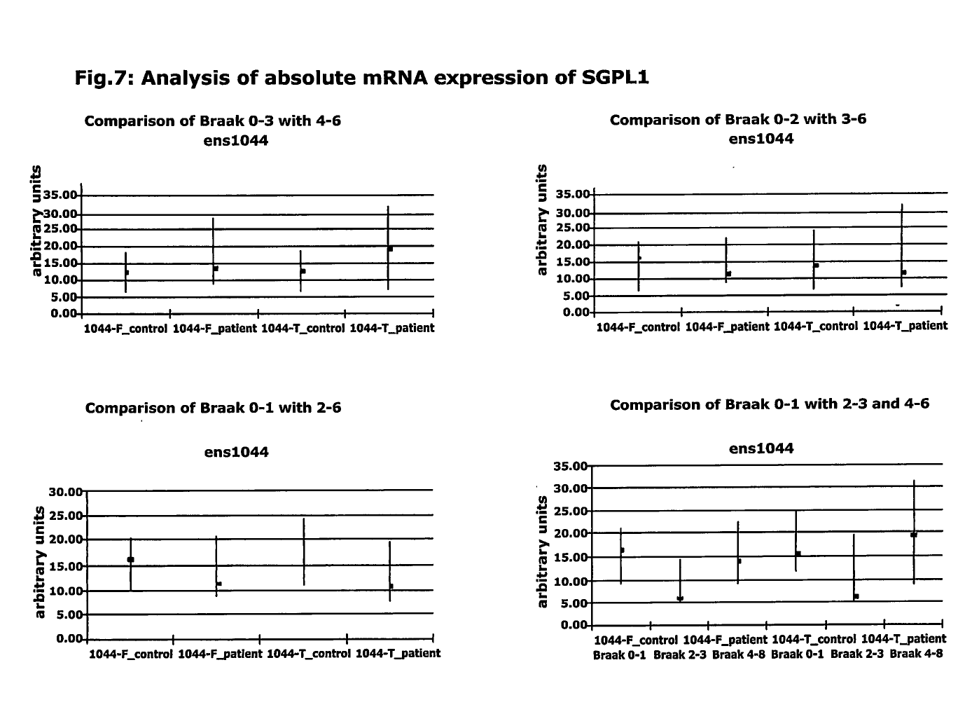

Second analysis used cyclophilin values from qPCR experiments of frontal cortex and inferior temporal cortex tissues from two different donors each, but different cDNA preparations from different time points were used. This analysis revealed a middle deviation of 29.2% from the median for comparison of absolute values and a middle deviation of 17.6% from the median for relative comparison. From this analysis it was concluded, that absolute values from qPCR experiments can be used, but the middle deviation from median should be taken into further considerations. A detailed analysis of absolute values for SGPL1 was performed. Therefore, absolute levels of SGPL1 were used after relative normalization with cyclophilin. The median as well as the 98%-confidence level was calculated for the control group (Braak 0-Braak 3) and the patient group (Braak 4-Braak 6), respectively. The same analysis was done redefining the control group (Braak 0-Braak 2) and the patient group (Braak 3-Braak 6) as well as redefining the control group (Braak 0-Braak 1) and the patient group (Braak 2-Braak 6). The latter analysis was aimed to identify early onset of mRNA expression differences between controls and AD patients. In another view of this analysis, three groups comprising Braak stages 0-1, Braak stages 2-3, and Braak stages 4-6, respectively, were compared to each other in order to identify tendencies of gene expression regulation as well as early onset differences. Said analysis as described above is shown in FIG. 7.

(iv) Immunoblotting:

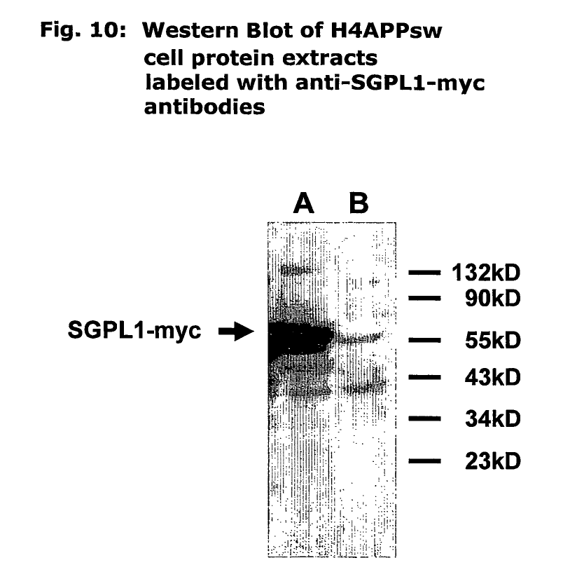

Total protein extract was obtained from H4APPsw cells expressing SGPL1-myc by homogenization in 1 ml RIPA buffer (150 mM sodium chloride, 50 mM tris-HCl, pH7.4, 1 mM ethylenediamine-tetraacetic acid, 1 mM phenylmethylsulfonyl flouride, 1% Triton X-100, 1% sodium deoxycholic acid, 1% sodium dodecylsulfate, 5 μg/ml of aprotinin, 5 μg/ml of leupeptin) on ice. After centrifuging twice for 5 min at 3000 rpm at 4° C., the supernatant was diluted five-fold in SDS-loading buffer. Aliquots of 12 μl of the diluted sample were resolved by SDS-PAGE (8% polyacrylamide) and transferred to PVDF Western Blotting membranes (Boehringer Mannheim). The blots were probed with rabbit polyclonal anti-myc antibodies (Mobitec, 1:500) followed by horseradish peroxidase-coupled goat anti-rabbit IgG antiserum (Santa Cruz sc-2030, diluted 1:5000) and developed with the ECL chemoluminescence detection kit (Amersham Pharmacia) (FIG. 10).

(v) Immunofluorescence Analysis (IF):

For the immunofluorescence staining of SGPL1 protein in cells, a human neuroglioma cell line was used (H4 cells) which stably expresses the human APP695 isoform carrying the Swedish mutation (K670N, M671L) (H4APPsw cells). The H4APPsw cells were transduced with a pFB-Neo vector (Stratagene, #217561, 6.6 kb) containing the coding sequence of SGPL1 (SGPL1 cds) (SEQ ID NO. 3, 1707 bp) (pFB-Neo-SGPL1cds, SGPL1 vector, 8280 bp, EcoR11/BamHI) and a myc-tag (pFB-Neo-SGPL1 cds-myc, SGPL1-myc vector, 8991 bp, EcoRI/XhoI) under the control of a strong CMV promotor. For the generation of the SGPL1-myc vector, the SGPL1cds-myc sequence was introduced into the EcoRI/XhoI restriction sites of the multiple cloning site (MCS) of the pFB-Neo vector. For transduction of the H4APPsw cells with the SGPL1-myc vector the retroviral expression system ViraPort from Stratagene was used.

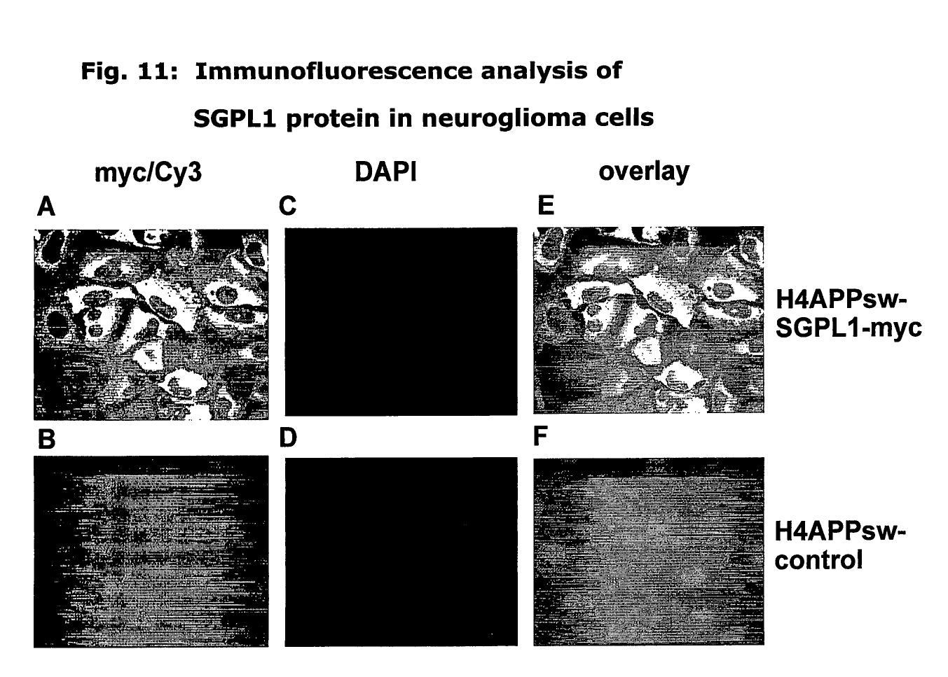

The myc-tagged SGPL1 over-expressing cells (H4APPsw-SGPL1-myc) were seeded onto glass cover slips in a 24 well plate (Nunc, Roskilde, Denmark; #143982) at a density of 5×10⁴ cells and incubated at 37° C. at 5% CO₂ over night. To fix the cells onto the cover slip, medium was removed and chilled methanol (−20° C.) was added. After an incubation period of 15 minutes at −20° C., methanol was removed and the fixed cells were blocked for 1 hour in blocking solution (200 μl PBS/5% BSA/3% goat serum) at room temperature. The first antibody (polyclonal anti-myc antibody, rabbit, 1:500, Mobitec) and DAPI (DNA-stain, 0.05 μg/ml, 1:1000) in PBS/1% goat serum was added and incubated for 1 hour at room temperature. After removing the first antibody, the fixed cells were washed 3 times with PBS for 5 minutes. The second antibody (Cy3-conjugated anti-rabbit antibody, 1:1000, Amersham Pharmacia, Germany) was applied in blocking solution and incubated for 1 hour at room temperature. The cells were washed 3 times in PBS for 5 minutes. Coverslips were mounted onto microscope slides using Permafluor (Beckman Coulter) and stored over night at 4° C. to harden the mounting media. Cells were visualized using microscopic dark field epifluorescence and bright field phase contrast illumination conditions (IX81, Olympus Optical). Microscopic images (FIG. 11) were digitally captured with a PCO SensiCam and analysed using the appropriate software (AnalySiS, Olympus Optical).

1. A method of diagnosing or prognosticating a neurodegenerative disease in a subject, or determining whether a subject is at increased risk of developing said disease, comprising determining a level and/or an activity of
(i) a transcription product of a gene coding for SGPL1, and/or
(ii) a translation product of a gene coding for SGPL1, and/or
(iii) a fragment, or derivative, or variant of said transcription or translation product,
in a sample obtained from said subject and comparing said level and/or said activity to a reference value representing a known disease or health status, thereby diagnosing or prognosticating said neurodegenerative disease in said subject, or determining whether said subject is at increased risk of developing said neurodegenerative disease.

2. A kit for diagnosing or prognosticating a neurodegenerative disease in a subject, or determining the propensity or predisposition of a subject to develop such a disease, said kit comprising:
at least one reagent which is selected from the group consisting of
(i) reagents that selectively detect a transcription product of a gene coding for SGPL1 and
(ii) reagents that selectively detect a translation product of a gene coding for SGPL1;
whereby the diagnosis or prognosis or determination of the propensity or predisposition to develop said neurodegenerative disease is determined by the steps of
(a) detecting in a sample obtained from said subject a level, or an activity, or both said level and said activity of a transcription product and/or of a translation product of a gene coding for SGPL1, and
(b) comparing said level or activity, or both said level and said activity of a transcription product and/or of a translation product of a gene coding for SGPL1 to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

3. A modulator of an activity and/or of a level of at least one substance which is selected from the group consisting of
(i) a gene coding for SGPL1,
(ii) a transcription product of a gene coding for SGPL1,
(iii) a translation product of a gene coding for SGPL1, and
(iv) a fragment, or derivative, or variant of (i) to (iii).

4. A recombinant, genetically altered non-human animal comprising a normative gene sequence coding for SGPL1 or a fragment, or a derivative, or a variant thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related diseases or disorders.

5. A method of developing diagnostics and therapeutics to treat neurodegenerative diseases, comprising screening, testing, or validating compounds, agents, and modulators using the recombinant, genetically altered non-human animal according to claim 4.

6. A method for screening for a modulator of neurodegenerative diseases, or related diseases or disorders of one or more substances selected from the group consisting of
(i) a gene coding for SGPL1,
(ii) a transcription product of a gene coding for SGPL1,
(iii) a translation product of a gene coding for SGPL1, and
(iv) a fragment, or derivative, or variant of (i) to (iii),
said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

7. A method of screening for a modulator of neurodegenerative diseases, or related diseases or disorders of one or more substances selected from the group consisting of
(i) a gene coding for SGPL1,
(ii) a transcription product of a gene coding for SGPL1,
(iii) a translation product of a gene coding for SGPL1, and
(v) a fragment, or derivative, or variant of (i) to (iii),
said method comprising:
(a) administering a test compound to a non-human test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched non-human control animal which is predisposed to developing or has already developed symptoms of a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the non-human test animal indicates that the test compound is a modulator of said diseases or disorders.

8. The method according to claim 7 wherein said non-human test animal and/or said control animal is a recombinant, genetically altered animal which expresses the gene coding for SGPL1, or a fragment, or a derivative, or a variant thereof, under the control of a transcriptional control element which is not the native SGPL1 gene transcriptional control element.

9. An assay for testing a compound, or a plurality of compounds to determine the degree of binding of said compounds to a SGPL1 translation product, or to a fragment, or derivative, or variant thereof, said assay comprising the steps of:
(i) adding a liquid suspension of said SGPL1 translation product, or a fragment, or derivative, or variant thereof, to a plurality of containers;
(ii) adding a detectable compound or a plurality of detectable compounds to be screened for said binding to said plurality of containers;
(iii) incubating said SGPL1 translation product, or said fragment, or derivative, or variant thereof, and said detectable compound or compounds;
(iv) measuring amounts of detectable compound or compounds associated with said SGPL1 translation product, or with said fragment, or derivative, or variant thereof; and
(v) determining the degree of binding by one or more of said compounds to said SGPL1 translation product, or said fragment, or derivative, or variant thereof.

10. The method of claim 1, comprising determining a level and/or an activity of a protein molecule of SEQ ID NO. 1, said protein molecule being a translation product of the gene coding for SGPL1, or a fragment, or derivative, or variant thereof.

11. The method of claim 6, wherein said screening is for a modulator of a protein molecule of SEQ ID NO. 1, said protein molecule being a translation product of the gene coding for SGPL1, or a fragment, or derivative, or variant thereof, wherein said modulator is a reagent or compound for preventing, or treating, or ameliorating Alzheimer's disease.

12. A method for detecting the pathological state of a cell in a sample obtained from a subject, comprising immunocytochemical staining of said cell with an antibody specifically immunoreactive with an immunogen, wherein said immunogen is a translation product of a gene coding for SGPL1, SEQ ID NO. 1, or a fragment, or derivative, or variant thereof, wherein an altered degree of staining or an altered staining pattern in said cell compared to a cell representing a known health status indicates a pathological state of said cell which relates to a neurodegenerative disease.

13. The method of claim 1, wherein said neurodegenerative disease is Alzheimer's disease.

14. The kit of claim 2, wherein said neurodegenerative disease is Alzheimer's disease.

15. The kit of claim 2, wherein said translation product is a protein molecule of SEQ ID NO. 1, said protein molecule being a translation product of the gene coding for SGPL1, or a fragment, or derivative, or variant thereof.

16. The recombinant, genetically altered non-human animal of claim 4, wherein said neurodegenerative disease is Alzheimer's disease.

17. The method of claim 6, wherein said neurodegenerative disease is Alzheimer's disease.

18. The method of claim 7, wherein said neurodegenerative disease is Alzheimer's disease.

19. The method of claim 7, wherein said screening is for a modulator of a protein molecule of SEQ ID NO. 1, said protein molecule being a translation product of the gene coding for SGPL1, or a fragment, or derivative, or variant thereof, wherein said modulator is a reagent or compound for preventing, or treating, or ameliorating Alzheimer's disease.

20. The assay of claim 9, wherein said detectable compound is a fluorescently labeled compound.

21. The method of claim 12, wherein said neurodegenerative disease is Alzheimer's disease.


SEQUENCE LISTING (15 sequences)

SEQ ID NO: 1 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKAYRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAAAWAALMHFGENGYVEATKQIIKTARFLKSELENIKGIFVFGNPQLSVIALGSRDFDIYRLSNLMTAKGWNLNQLQFPPSIHFCITLLHARKRVAIQFLKDIRESVTQIMKNPKAKTTGMGAIYGMAQTTVDRNMVAELSSVFLDSLYSTDTVTQGSQMNGSPKPH

SEQ ID NO: 2 - DNA, 5741 bp, Artificial Sequence
  Description of Artificial Sequencenucleotide sequence of human SGPL1 cDNA
gcggctgccgggcctccaatctcggcggcggcggcggcaacaggggagcctgggtctcgcggcctgcgagtccgtcgcgtgctgagggagacgcaggaggtggagccggccgggtgctcgagggaaggagactggaagctggttccggcgtgaggagagtctgaaaaaggggagcgcggagaggaggctggaagaggaagatgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactgaacttggaccctttctagtctcaaggggattccagccttcagaaggttcttgggatatggaacaggccgtgcacaactttgacatctggtcttgctccatagagcacaactcaagatagaccatgagacagcttgagcctcaggattcttgttcttcctcttatcttccttttgtggtttttaatttgaagaccccagagaattccattacataatgattttgcccttgttataaatgttaccctaggaattgttttaaccatttccttttctaaactctctagctttcaactttacttaaacattgtgtggtagctctgacctgtcctgattctttagagaagctggggtacagtttatgagatagctagagcttctttgttatctcaggcaggaggcgtttacataacagatgtttcctcagctgggtgtgaggtatactctaagcaggaggctttttcagccttctctctcttttttttttttttttttttttgagatggaattttgctcttttgcccagtctggagtgcagtggcatgatctcagctcactgcaacctccacccactgggttcaagcgattcttctgcctcagcctcccgagtagctgggattaccggcacccaccaccacgcctggctaatttttcaattttctttttcagtagagacgggttcaccgtgttggccaggctggtcttgaactcctgacctcaggtgatacccgcccccccgcctcagcctcccaaagtgctgggattacaggcgtgagccaccgtgcctggccctgtctctcttaagagtaggttcattgtctgtcttagagtcacttctattgcaactcattttctttttccagggcacagatcgaccaagctgccgttccctattctgcaggacaggactattctagcatacctgcttcgtccacccaggcagggtttggggtggtctcttctgtgcctgcagtccccatttgacacttggttgccaccatctttggagattattgtttggaatgatgcttccattggctttttcttgttaccatggactaggaagaaaacatggtttccaaataatctgggagcttttggccatggtgccgccttcctgaattggcagtggtcagagcacacctgaaccctatcctgggctggtgatgagcagaaatcagacctttttctatgcttttttgaatatcagagtaggatgaacacccagattcaaatatgtcaccaaagttggtggtggtccttccctgcacccttgcgttaagccattatgtaatgaaaatgtgtttgcttgaaggaacagctcaaagcaccttcacaagttgccttgacttaccctaggtgggtgtgaaagagcacccgtagcaaggaaaattttctctattagtgtgttcttctgcctcttcccccttgattcagctttcagaggtactatggcagttttgcctcaggtgctgaacatttctcagccctggctaaaagggagcagcacagggagagaaacaggataggaaagcagaatggcgagcagcctatggcccagggcctgtaatcccttcccaagactagctgctcagggtggtgcagggacaggaccagaccctgcgcctatttcctgccttctttcccctatagggaactctgtaggctgagccactgtcctgctcttatgacattatatcttgtgcctttctcctcagcagtgagcagtgagctactcctggcccaggccctaggggaaatggatcagtctttgaggtttctatttggggaggggagtacttaagatgagtcaaaagacactttcctctgttccattccccatctcagggactcctgaatattcagcctctccaggctggtgtcttctagtttcccccactgggaatgctggctgggagagccatgactaccagacttttcctcaggctccttggcatgttagtctgaattgttcttgagcactgtactactgacccaacaactgtgactagctggccacgccattcagggctggtgtggcatttatgtgtgtgtgtgtgtgtgtgtgtttttcctgtttgcccagcagtgcattgtgggttccaagagtgggtagtgtgtgtatgtgtgtgtgtcagagggagacctggcaggcacctctttgagagtagctgtggtcagagctgtttggtcagtgcattatgttgaatgaggtccaggaacccagagccacccagcagacaccactgtggcttgccagctgccaagatggagaagcatgtgcccctgtagagcgtctccccagaaccagaccccgagccactcgcttcctctgtgctgtgacaacattggtgccaggggagatggtgtttttcaaagggacctactgtagccactttaatttacaattaagagccttagtttgacttaacacttttgtaggcttttcattgtgtatttttgtgtatgtgtgcatatagcagctactctgtagcagaggtgggtagagacacttaatagtatcatgtcgcatgcagatgtcacatcggcctctgcaaaaactgtactgtcttgtttctgcattagacttaagtagtcatgtgaatatactgctatgtcacttttaatattacgagttttatacttggaaaatggtacttgcttcttttaaatctctgtcttctctaacctcccccttcccatttcaatgctcccttcctaatttcagcaataatctcaaaaagcaattaaatagttaaatgaccctaattgtaattactgtggatggttgcattcatttgattacttgggcacacacgagatgacaaatggggcagtggccatgcttgaatgggctcctggtgagagattgccccctggtggtgaaacaatcgtgtgtgcccactgataccaagaccaatgaaagagacacagttaagcagcaatccatctcatttccaggcacttcaataggtcgctgattggtccttgcaccagcagtggtagtcgtacctatttcagagaggtctgaaattcaggttcttagtttgccagggacaggccctatcttatatttttttccatcttcatcatccacttctgcttacagtttgctgcttacaataacttaatgatggattgagttatctgggtggtctctagccatctgggcagtgtggttctgtctaaccaaagggcattggcctcaaaccctgcatttggtttaggggctaacagagctcctcagataatcttcacacacatgtaactgctggagatcttattctattatgaataagaaacgagaagtttttccaaagtgttagtcaggatctgaaggctgtcattcagataacccagcttttccttttggcttttagcccattcagactttgccagagtcaagccaaggattgcttttttgctacagttttctgccaaatggcctagttcctgagtacctggaaaccagagagaaagaggatccaggatgtacttggatgaggaggcctggcttatctaggaagtcgtgtctggggtgcttattgctgctccatacagctgtacgtcagccccttggccttctctgtaggttcttggcagcaatgagcagctttcactcagtgacacaagtaattactgagtcctaatttgatagccaccaactgtacctgggtaggcaaagtcagatttttgagaacctttttcctgatttgaagttttaattaccttattttcttttatgctttcctctgtcttgtaatcttttctcttcttaatatccttccctataatttcaattatttggattaattttagaataaacctatttatttct

SEQ ID NO: 3 - DNA, 1707 bp, Homo sapiens
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactga

SEQ ID NO: 4 - DNA, 21 bp, Artificial Sequence
  Description of Artificial Sequenceprimer for the human SGPL1 gene
tgcccactgataccaagacca

SEQ ID NO: 5 - DNA, 21 bp, Artificial Sequence
  Description of Artificial Sequenceprimer for the human SGPL1 gene
agtgcctggaaatgagatgga

SEQ ID NO: 6 - DNA, 20 bp, Artificial Sequence
  Description of Artificial Sequenceprimer for the human cyclophilin B gene
actgaagcactacgggcctg

SEQ ID NO: 7 - DNA, 19 bp, Artificial Sequence
  Description of Artificial Sequenceprimer for the human cyclophilin B gene
agccgttggtgtctttgcc

SEQ ID NO: 8 - DNA, 20 bp, Artificial Sequence
  Description of Artificial Sequenceprimer for the human ribosomal protein S9 gene
ggtcaaatttaccctggcca

SEQ ID NO: 9 - DNA, 22 bp, Artificial Sequence
  Description of Artificial Sequenceprimer for the human ribosomal protein S9 gene
tctcatcaagcgtcagcagttc

SEQ ID NO: 10 - DNA, 19 bp, Artificial Sequence
  Description of Artificial Sequenceprimer for the human beta-actin gene
tggaacggtgaaggtgaca

SEQ ID NO: 11 - DNA, 19 bp, Artificial Sequence
  Description of Artificial Sequenceprimer for the human beta-actin gene
ggcaagggacttcctgtaa

SEQ ID NO: 12 - DNA, 20 bp, Artificial Sequence
  Description of Artificial Sequenceprimer for the human GAPDH gene
cgtcatgggtgtgaaccatg

SEQ ID NO: 13 - DNA, 21 bp, Artificial Sequence
  Description of Artificial Sequenceprimer for the human GAPDH gene
gctaagcagttggtggtgcag

SEQ ID NO: 14 - DNA, 21 bp, Artificial Sequence
  Description of Artificial Sequenceprimer for the human transferrin receptor TRR gene
gtcgctggtcagttcgtgatt

SEQ ID NO: 15 - DNA, 23 bp, Artificial Sequence
  Description of Artificial Sequenceprimer for the human transferrin receptor TRR gene
agcagttggctgttgtacctctc